(19) United States
(12) Patent Application Publication
(10) Pub. No.: US 2025/0009728 A1
(43) Pub. Date: Jan. 9, 2025

(54) BENZIMIDAZOLE DERIVATIVES COMPOSITIONS

(71) Applicant: Hikma Pharmaceuticals USA Inc., Berkeley Heights, NJ (US)

(72) Inventors: Katarina Alilovic, Zagreb (HR); Tina Gjoni, Zagreb (HR); Anita Mocnik, Zagreb (HR)

(73) Assignee: Hikma Pharmaceuticals USA Inc., Berkeley Heights, NJ (US)

(21) Appl. No.: 18/894,254

(22) Filed: Sep. 24, 2024

Related U.S. Application Data
(63) Continuation of application No. 18/166,020, filed on Feb. 8, 2023, now abandoned.
(60) Provisional application No. 63/309,296, filed on Feb. 11, 2022.

(51) Int. Cl. A61K 31/4439; A61K 9/08; A61K 47/18; A61K 47/40

(52) U.S. Cl. CPC A61K 31/4439; A61K 9/08; A61K 47/183; A61K 47/40

(57) ABSTRACT

The present disclosure relates to compositions including benzimidazole derivatives. Specifically, the disclosure is generally directed to storage stable formulations of benzimidazole derivatives.

### CROSS-REFERENCE TO RELATED APPLICATIONS

This application is a continuation of U.S. Application Ser. No. 18,166,020, filed on Feb. 8, 2023, which claims priority to U.S. Provisional Application 63/309,296 filed on Feb. 11, 2022, which is incorporated herein by reference in its entirety.

### FIELD OF THE INVENTION

The present disclosure relates to compositions comprising benzimidazole derivatives. Specifically, the disclosure is generally directed to storage stable formulations of benzimidazole derivatives.

### BACKGROUND

One particular class of benzimidazole derivatives comprises at least one sulfinyl group bridging benzimidazole and pyridine rings. That particular class comprises proton pump inhibitors. Proton pump inhibitors (hereinafter “PPI”) are a class of medicaments whose main action is a pronounced and long-lasting reduction of stomach acid production.

Proton pump inhibitors (PPIs) are used in the treatment of a number of conditions which require reduction in acid production by proton pump inhibition, such as Gastroesophageal Reflux Disease Associated (including patients with a History of Erosive Esophagitis), healed reflux esophagitis, Pathological Hypersecretion Including Zollinger-Ellison Syndrome, Erosive Esophagitis, upper gastrointestinal bleeding, relapse of gastric and duodenal ulcers, NSAID-associated gastric and duodenal ulcers and Helicobacter pylori (H. pylori) eradication in peptic ulcer disease.

Typically, omeprazole, esomeprazole, lansoprazole, pantoprazole, dexlansoprazole and rabeprazole are formulated as tablets or capsules for oral administration. An alternative to the above formulations is the provision of the active ingredients as solids for suspension or reconstitution/dilution immediately before use. Such preparation before immediate administration needs a strict control in the way it is prepared. As a result, there is a chance that the medicine may be rendered ineffective if not prepared correctly.

Pantoprazole is also currently available on the market as a lyophilized drug product, i.e., PROTONIX IV®, for injection. This dosage form must be reconstituted before administration by injection such as parenteral injection. This generally requires a number of steps, including dissolution (or reconstitution) of the lyophilized drug product in a suitable liquid for injection, such as Water for Injection, 0.9 percent Sodium Chloride Injection, or other fluids known in the art for this purpose to yield a concentrated solution suitable for intravenous administration. Complete dissolution into the liquid typically requires physical manipulation, e.g., swirling or shaking, and can be time consuming. Careful dilution of the reconstituted (concentrated) solution must then be performed with the correct volume of suitable diluent, such as 0.9 percent Sodium Chloride Injection or 5 percent Dextrose Injection or Lactated Ringer's Injection, to ensure the correct concentration for administration. Incomplete dissolution during the reconstitution step and/or incorrect dilution during the subsequent dilution step can lead to improper dosing, a significant component of medical error. The use of an incorrect reconstitution fluid and/or diluent can also lead to dosing errors. Such lyophilized dosage forms have been claimed in, for example, U.S. Pat. Nos. 6,780,881, 7,351,723 and 8,754,108.

A further limitation of lyophilized drug products which must be reconstituted with a suitable liquid for injection is the relatively short shelf-life which is associated with such preparations after reconstitution and/or dilution. These reconstituted drug products are often only labelled as usable for only a few hours after reconstitution and/or dilution.

For example, reconstituted or diluted PROTONIX IV® solutions are only stable for up to 24 hours at room temperature from the time of initial reconstitution.

It was also found that benzimidazole derivatives are not very physically stable in the presence of liquid. For instance, it was found that liquid formulations comprising pantoprazole tend to precipitate, which makes it difficult to handle and store, and consequently makes it difficult to scale up method of preparation of liquid pantoprazole to an industrial level. It was also found that pantoprazole in liquid forms unclear composition with particles which cannot be used for IV injections.

Therefore, there was a need for stable, aqueous solutions of the benzimidazole derivatives in accordance with the present disclosure which will be stable for certain prolonged periods of time on storage conditions.

### SUMMARY

The present disclosure is generally directed to storage of stable formulations of benzimidazole derivatives. In one aspect, present disclosure is directed to precipitate-free formulations of benzimidazole derivatives.

In one aspect, benzimidazole derivatives contain at least one sulfinyl group bridging the benzimidazole and pyridine rings.

In one aspect, benzimidazole derivatives contain one sulfinyl group bridging the benzimidazole and pyridylmethyl moiety.

In one aspect, the benzimidazole derivatives are proton pump inhibitors.

Examples for these benzimidazole derivatives are omeprazole, lansoprazole, esomeprazole, dexlansoprazole, rabeprazole, and pantoprazole.

In one aspect, the benzimidazole derivative is pantoprazole.

In one aspect, the composition of benzimidazole derivatives is a semi-liquid, liquid or aqueous composition.

In one aspect, composition is a liquid.

In one aspect, composition is an aqueous composition.

According to the present disclosure, the composition of benzimidazole derivatives comprises at least one cyclodextrin.

In one aspect, the composition of benzimidazole derivative optionally comprises at least one buffer which is capable of maintaining the pH of the composition between 8.5 and 12 during a certain period of time.

In one aspect, liquid composition of benzimidazole derivative is a ready-to-use composition. In one aspect, the composition in stored in a single unit container.

According to the present disclosure, a method for producing precipitate free compositions of benzimidazole derivatives is provided.

Pharmaceutical formulations according to the present disclosure have enhanced storage stability. Therefore, the compositions disclosed herein can be advantageously stored for relatively long periods of time.

According to the present disclosure, a method of producing benzimidazole derivative formulations is provided.

Pharmaceutical formulations according to the present disclosure may be used to treat a patient with a gastroesophageal reflux disease (GERD), ulcers of the stomach and duodenum, and Zollinger-Ellison Syndrome by administering the pharmaceutical formulation.

It is to be understood that both the foregoing description and the following further description are exemplary and explanatory only and are not restrictive of the claims.

### DETAILED DISCLOSURE (INCLUDING DEFINITIONS)

The present disclosure is generally directed to storage stable formulations of benzimidazole derivatives.

In one aspect, benzimidazole derivative formulations are precipitate free benzimidazole formulations.

In one aspect, formulations of benzimidazole derivatives are pharmaceutically acceptable formulations.

In one aspect, formulations of benzimidazole derivatives are ready-to-use pharmaceutical compositions of benzimidazole derivatives.

Examples for these benzimidazole derivatives are omeprazole, lansoprazole, esomeprazole, dexlansoprazole, rabeprazole, and pantoprazole or pharmaceutically acceptable salts thereof.

In one aspect, the benzimidazole derivative is pantoprazole or its pharmaceutically acceptable salt.

In one aspect, the composition of benzimidazole derivatives is a semi-liquid, liquid or aqueous composition.

In one aspect, the composition of benzimidazole derivatives is liquid.

In one aspect, the composition of benzimidazole derivatives is aqueous composition.

In one aspect, the disclosure relates to aqueous compositions comprising pantoprazole or its pharmaceutically acceptable salt.

In one aspect, the pantoprazole is a sodium salt. In another aspect, the pantoprazole is pantoprazole sodium sesquihydrate.

In one aspect, the aqueous composition is a ready-to-use pantoprazole compositions.

As used herein, the terms “composition” and “formulation” are used interchangeably.

By the term “aqueous composition”, “aqueous solution” or “aqueous” is understood any composition in which water is present in or above 50% v/v, such as, e.g., a composition comprising from 50% v/v to 99.5% v/v water, from 50% v/v to 90% v/v, from 60% v/v to 85% v/v, from 70% v/v to 80% v/v water. Accordingly, aqueous compositions include compositions comprising 50% v/v or more, 60% v/v or more, 70% v/v or more, 75% v/v or more, 80% v/v or more, 85% v/v or more, 90% v/v or more, 95% v/v or more or 99% v/v water or more.

In one aspect, an aqueous composition of benzimidazole derivative comprises 90% or more water.

In one aspect, an aqueous composition of pantoprazole comprises 90% or more water.

By terms “pharmaceutical composition” or “pharmaceutically acceptable composition” as used herein, is meant any composition suitable and intended for in vivo use, for example administration to a patient or a subject. As used herein, the terms “patient” and “subject” are interchangeable and refer to any human or animal individual who is receiving a composition as described herein.

The compositions may be in the form of a ready-to-administer or a ready-to-use composition.

The term “ready-to-use” includes liquid preconcentrates which require a single step of preparation with a suitable diluent such as water, suitable diluents for injection, flavored solutions or juices before administration. A “ready-to-use” composition is distinguished from lyophilized products because a “ready-to-use” composition does not require a step of reconstitution to form a liquid, aqueous composition.

A “ready-to-administer” composition is synonymous with “ready-to-infuse” or “ready-to-inject” and is not to be read as the term “ready-to-use” composition. A “ready-to-administer” composition is suitable for administration directly to the patient and does not require any dilution steps.

The term “ready-to-administer” is also distinguished from lyophilized products that require two steps, a first step of reconstitution to form a preconcentrate and then a second step where the preconcentrate is subjected to dilution with a liquid infusion fluid.

In one aspect, compositions of benzimidazole derivatives comprise at least one cyclodextrin.

In one aspect, compositions of pantoprazole comprise at least one cyclodextrin. In one aspect, liquid compositions of pantoprazole comprise at least one alpha, gamma or beta cyclodextrin.

In one aspect, liquid compositions of benzimidazole derivatives comprise at least one beta cyclodextrin.

In one aspect, liquid compositions of pantoprazole comprise at least one beta cyclodextrin.

In one aspect, cyclodextrins are 2-hydroxypropyl-beta-cyclodextrin (HPBCD) and sulfobutylether-beta-cyclodextrin (SBEBCD).

In one aspect, the beta cyclodextrin is HPBCD.

Hydroxypropyl-beta-cyclodextrin is a partially substituted poly(2-hydroxpropyl) ether of beta-cyclodextrin. The degree of substitution is normally expressed as a molar substitution (MS) value, which is the number of hydroxypropyl groups per one glucose unit.

In one aspect, the molar substitution of HPBCD 0.4-1.5.

In one aspect, the molar substitution of HPBCD 0.5-1.1.

In another aspect, the molar substitution of HPBCD is 0.6-1.0.

In another aspect, the molar substitution of HPBCD is 0.5, 0.6, 0.7, 0.8, 0.9, 1.0 or 1.1.

In one aspect, the benzimidazole derivative and at least one cyclodextrin are in molar ratio of 1:1 to 1:14.

In one aspect, pantoprazole and at least one cyclodextrin are in a molar ratio of 1:1 to 1:14.

In other aspect, pantoprazole and at least one cyclodextrin are in a molar ratio of 1:2 to 1:9. In other aspect, pantoprazole and at least one cyclodextrin are in a molar ratio of 1:3 to 1:6.

In one aspect, benzimidazole derivative and at least one cyclodextrin are in a molar ratio of 1:1, 1:2, 1:3, 1:4, 1:5, 1:6, 1:7, 1:8, 1:9, 1:10, 1:11, 1:12, 1:13, or 1:14.

In one aspect, pantoprazole and at least one cyclodextrin are in a molar ratio of 1:1, 1:2, 1:3, 1:4, 1:5, 1:6, 1:7, 1:8, 1:9, 1:10, 1:11, 1:12, 1:13, or 1:14.

In one aspect, the pH of the formulation is from 8.5 to 12.0.

In one aspect, the pH of the formulation is from 8.5 to 10.5.

In one aspect, the pH of the formulation is from 10.0 to 12.0.

In one aspect, the pH of the formulation is from 10.5 to 12.0.

In one aspect, the pH of the formulation is from 10.5 to 11.5.

In one aspect, the pH of the formulation is 8.5, 9.0, 9.5, 10.0, 10.2, 10.5, 10.8, 11.0, 11.2, 11.5, 11.7, 12.0.

“pH” is the conventional measurement unit of hydrogen ion activity in a solution at room temperature, unless another temperature is specified.

As used herein, the term “pH” in compositions is defined as ±0.3 of the numerical value or range in question.

In one aspect, compositions according to the present disclosure comprise at least one buffer which is capable of maintaining the pH of the composition from 8.5 to 12 during a certain period of time.

In one aspect, the buffer is capable of maintaining the pH of the composition from 10.0 to 12.0.

Buffer capacity may be calculated with any method know to the person skilled in the art. In one aspect, buffer capacity may be calculated by using software like CurTiPot™ or by using the Henderson-Hasselbach equation.

In one aspect, the composition is comprises at least one buffer having at least one ionizable group with a pKa in the range 7.0 to 14 and which pKa is within 2 pH units of the pH of the composition.

Buffers suitable for use in the pharmaceutical compositions described herein include, but are not limited to, pharmaceutically acceptable salts and acids of phosphate, carbonate, meglumine, ammonium, glycine and borate.

In one aspect, the buffer is selected from a carbonate or phosphate buffer or mixture thereof.

In one aspect, the buffer is a carbonate buffer. By term carbonate buffer is meant any buffer containing carbonate ions.

In one aspect, the carbonate buffer is a mixture of carbonate and bicarbonate ions. In one aspect, the carbonate buffer is a mixture of sodium carbonate and sodium bicarbonate.

In one aspect, the buffer is a phosphate buffer. By term phosphate buffer is meant any buffer containing phosphate ions. A non-exhaustive list of phosphate ions is phosphate, hydrogen phosphate, dihydrogen phosphate, and mixture thereof.

In one aspect, the phosphate buffer is mixture of hydrogen phosphate and dihydrogen phosphate ions. In one aspect, the phosphate buffer is mixture of sodium hydrogen phosphate and sodium dihydrogen phosphate.

In one aspect, the concentration of at least one buffer is from 20 mM to 200 mM.

In one aspect, the concentration of at least one buffer is from 20 mM to 50 mM.

In one aspect, the concentration of at least one buffer is from 50 mM to 100 mM.

In one aspect, the concentration of at least one buffer is 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM or 200 mM.

In one aspect, the concentration of carbonate buffer is 20 mM.

In one aspect, the concentration of carbonate buffer is 50 mM.

In one aspect, the concentration of phosphate buffer is 100 mM.

In one aspect, the concentration of phosphate buffer is 200 mM.

It was found that, when pantoprazole is formulated in formulations according to the present disclosure, degradation product formation is retarded, and accordingly, such formulations exhibit prolonged chemical and physical stability and provide more flexible storage conditions and handling when stored under refrigerated conditions, i.e., at a temperature of from 2° C. to 8° C. Furthermore, the pharmaceutical formulation in accordance with the present disclosure has improved stability under room temperature conditions. Further, the pharmaceutical formulation in accordance with the present disclosure has improved stability at a temperature of 40° C.

The term “stable” means that the pharmaceutical compositions meets one or more of the following criteria:
(i) The pharmaceutical composition exhibits an acceptable amount of active ingredient degraded after a certain period compared to the amount of pantoprazole present at the beginning of the period; and/or
(ii) the pharmaceutical composition exhibits an acceptable amount of impurities being formed after a certain period compared to the amount of impurities present at the beginning of the period; and/or
(iii) the pharmaceutical composition retains a pharmaceutically desirable appearance such as clarity, improved color, and no visible particles (i.e., precipitate free or particle free composition). Visual inspection for visible particles may be performed as follows: the container under inspection is gently swirled and inverted, ensuring that no air bubbles are produced, and inspected during certain period of time (approximately 5 to 20 sec) with naked eye and/or under the magnifier. Visual inspection for change of color may be performed as follows: The container is inspected by eye and a color is assigned to the composition. The color may also be determined by a UV/VIS spectrometer and the difference in color between two samples may be expressed as delta E.

Formulations according to the present disclosure showed surprising stability for a reasonable period of time, when stored at a temperature of from 2° C. to 8° C., such as, e.g., at a temperature of 2° C., 3° C. or less, 4° C. or less, 5° C. or less, 6° C. or less, 7° C. or less or 8° C. or less.

In one aspect, the compositions are stable for at least 1 month, at least 2 months, at least 3 months, at least 6 months, at least 9 months, or at least 12 months, when stored at temperature of from 2° C. to 8° C.

In one aspect, a liquid formulation according to the present disclosure is stable under room temperature conditions for a certain period of time.

By the term “room temperature” used herein, is a room temperature which is from 20° C. to 25° C.

In one aspect, the compositions are stable for at least 7 days, 14 days, at least 1 month, at least 2 months, at least 3 months, at least 6 months, at least 9 months, or at least 12 months, when stored at room temperature conditions.

In one aspect, the compositions are stable for at least at least 3 months when stored at room temperature conditions.

Formulations according to the present disclosure showed surprising stability for a reasonable period of time, when stored at a temperature of 40° C.

In one aspect, the compositions have been stable for at least 24 h, at least 36 h, at least 48 h, at least 72 h, at least 1 week (7 days), at least 14 days, at least 1 month, at least 2 months, at least 3, at least 6 months, when stored at temperature of 40° C.

In one aspect, “stability” may be defined by the amount of total or individual impurities in the formulation after a certain period of time. Stability may also be defined by increase of total or individual impurities generated after a suitable period of time.

The stability may be determined by measuring the amount of individual impurity in the formulation according to the present disclosure after a predetermined time period, preferably expressed as a percentage, for example as a peak-area percentage of a chromatogram.

By the term “impurity” as used herein is meant an impurity of the active pharmaceutical ingredient in the pharmaceutical formulation.

By the individual impurity related to pantoprazole, it is predominantly meant the impurity characterized as 5-difluoromethoxy-2-[[(3,4-dimethoxy-2-pyridinyl)methyl]sulphonyl]-1H-benzimidazole, known also as related compound A or Impurity A. Structure of Impurity A is shown below as FIG. 1.

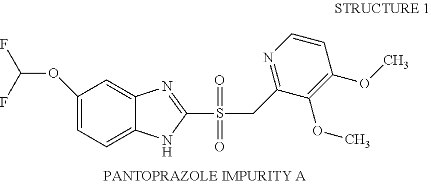 [SMILES: COc1ccnc(CS(=O)(=O)c2nc3cc(OC(F)F)ccc3[nH]2)c1OC]

In one aspect, the composition has less than 0.05% of Impurity A after at least 7 days at 40° C.

In one aspect, the composition has less than 0.08% of Impurity A after at least 3 months at 25° C.

In one aspect, the composition has less than 0.05% of Impurity A after at least 3 months at 25° C.

As used herein, “stable” is defined as no visible particles (free of particles or particles/precipitate free) in the pharmaceutical formulation after a predetermined time period. It has been discovered that benzimidazole derivatives having at least one sulfinyl group bridging the benzimidazole and pyridine rings are susceptible to precipitation. According to the present disclosure, the precipitation of benzimidazole derivatives having at least one sulfinyl group bridging benzimidazole and pyridine rings is retarded.

In one aspect, the composition is free of particles for at least 7 days at 40° C.

In one aspect, the composition is free of particles for at least 3 months at 25° C.

As used herein, “stable” is defined as no more than 10% of assay decrease/drop of active ingredient in the pharmaceutical formulation, determined by UPLC analysis.

For example, a stable composition can be one which has no more than 1%, 2%, 3%, 4%, 5%, 6%, 7%, 8%, 9%, 10%, of assay decrease/drop for active pharmaceutical ingredient after a predetermined time period.

In an aspect, a stable composition can be one which has no more than 8% of assay decrease/drop for individual active pharmaceutical ingredient after a predetermined time period.

In one aspect, the concentration of pantoprazole is from 0.2 mg/ml to 40 mg/ml.

In one aspect, the concentration of pantoprazole is from 0.2 mg/ml to 10 mg/ml.

In one aspect, the concentration of pantoprazole may be 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml.

In one aspect, the concentration of pantoprazole is 0.4 mg/ml.

In one aspect, the concentration is of pantoprazole 4 mg/ml.

In one aspect, the composition of pantoprazole is a ready to administer composition.

In one aspect, the ready-to-use composition of pantoprazole may be diluted prior to administration. In one aspect, the diluent may be some of standard diluents for parenteral or oral use. In one aspect, the diluent may be selected from 0.9% sodium chloride for injection, dextrose 5% injection, Ringer's solution or other suitable diluents.

In one aspect, the composition comprises at least one amino acid, its pharmaceutically acceptable salt or derivative. In one aspect, the composition comprises at least two amino acid, its pharmaceutically acceptable salt or derivative.

In one aspect, the amino acid is arginine, tryptophane, phenylalanine, tyrosine, proline, taurine, lysine, histidine, glutamine, glutamate, or a pharmaceutically acceptable salt or derivative.

In one aspect, the benzimidazole derivative and at least one amino acid are in a molar ratio of 1:0.5 to 1:20.

In one aspect, the benzimidazole derivative and at least one amino acid are in a molar ratio of 1:0.5, 1:1, 1:1, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20.

In one aspect, pantoprazole and at least one amino acid are in a molar ratio of 1:0.5 to 1:20.

In one aspect, pantoprazole and at least one amino acid are in a molar ratio of 1:0.5 to 1:10.

In one aspect, pantoprazole and at least one amino acid are in a molar ratio of 1:2 to 1:6.

In one aspect, pantoprazole and at least one amino acid are in a molar ratio of 1:5.

In one aspect, the amino acid is taurine, its pharmaceutically acceptable salt or derivative.

In one aspect, the amino acid is arginine, its pharmaceutically acceptable salt or derivative.

In one aspect, the amino acid is tryptophane, its pharmaceutically acceptable salt or derivative.

In one aspect, the composition comprises at least one sugar or sugar alcohol.

In one aspect, the sugar may be sucrose, trehalose.

In one aspect, the sugar is sucrose.

In one aspect, pantoprazole and at least one sugar are in a molar ratio of 1:2 to 1:10.

In one aspect, pantoprazole and at least one sugar alcohol are in a molar ratio of 1:0.5 to 1:5.

In one aspect, the sugar alcohol is sorbitol.

In one aspect, the composition comprises sodium iodide, dietanolamine, creatinine, and/or KCl.

The pH of the solutions can be adjusted in any suitable manner. The pH may be adjusted with one or more pH adjusting agents, which may be selected from mineral acids, organic acids, weak and strong bases, and salts and derivatives thereof. Examples of agents include hydrochloric acid, phosphoric acid, sulfuric acid, acetic acid, succinic acid, lactic acid, citric acid, phenolic acids, sodium hydroxide, ammonium hydroxide, sodium bicarbonate, or similar.

In one aspect, the pH is adjusted with sodium hydroxide and hydrochloric acid.

In one aspect, the composition is not reconstituted from a lyophilized composition of benzimidazole derivatives.

In one aspect, the composition is not reconstituted from a lyophilized composition of pantoprazole.

In one aspect, the aqueous pharmaceutical solution of the pantoprazole is administered parenterally. Suitable methods for parenteral administration include, but are not limited to, administering a sterile aqueous preparation to the human in need thereof. In one aspect, the composition is in a unit-dose form. In another aspect, the composition is in a multi-dose form.

The terms “treat,” “treatment,” and “treating” refer to one or more of the following preventing, relieving or alleviating at least one symptom of certain esophagus, stomach and intestinal problems in a subject.

In one aspect, compositions may be administered by parenteral routes, including, subcutaneous, intramuscular, intravenous, intra-atrial, or intra-arterial continuous infusion to a patient.

In one aspect, the compositions may be administered by an oral route.

In one aspect, the present disclosure provides a method of treating humans by administering an effective dose of the benzimidazole derivative by a parenteral route.

In one aspect, the present disclosure provides a method of treating humans by administering a ready to use, effective dose of a pantoprazole formulation by IV injection.

In one aspect, the present disclosure provides a method of treating humans by diluting a ready to use effective dose of a pantoprazole formulation and administering said dilution by IV infusion.

In one aspect, the present disclosure provides a method for administering pantoprazole to a subject in need thereof, the method comprising drawing a composition comprising pantoprazole from a ready to use pharmaceutical product into a container, diluting with a diluent to achieve a desired concentration, and administering the composition into the subject using an IV infusion.

In one aspect, the present disclosure provides a method for administering pantoprazole to a subject in need thereof, the method comprising drawing a composition comprising pantoprazole from a ready to use pharmaceutical product into a syringe and injecting the composition into the subject using the syringe.

In one aspect, administration does not involve reconstitution of a lyophilized composition of benzimidazole derivative.

In one aspect, the liquid formulation of pantoprazole in packaged into container. In one aspect, the container is single unit dose container. In one aspect, the container is a single unit dosage container for IV administration.

The liquid pantoprazole formulations as described herein are useful for treatment of gastroesophageal reflux disease (GERD), ulcers of the stomach and duodenum, and Zollinger-Ellison Syndrome.

In still a further aspect, the present disclosure provides method of treating a subject in need thereof by administering an effective dose of the liquid pantoprazole formulation as disclosed herein.

While the above description contains many specific aspects, these should not be construed as limitations on the scope of the disclosure, but rather as an example of one aspect thereof. Accordingly, the scope of the disclosure should be determined not by the aspects illustrated, but by the appended claims and their equivalents.

All of the numbers used herein are modified by the term “about.” This means that each number includes minor variations as defined±10% of the numerical value or range in question.

Typically, ready to use pharmaceutical compositions have a tonicity from 250 to 350 mOsm/kg.

In some aspects, the ready to use pharmaceutical compositions further comprise one or more tonicity agents. Suitable tonicity agents for use in ready to use pharmaceutical compositions include, but are not limited to, anhydrous or hydrous forms of sodium chloride, dextrose, sucrose, xylitol, fructose, glycerol, sorbitol, mannitol, potassium chloride, mannose, calcium chloride, magnesium chloride and other inorganic salts.

If not otherwise stated, calculations of molar ratio or concentrations of pantoprazole in the present disclosure are done based on pantoprazole free base.

### Methods

The compositions described herein can be made by prepared in a number of different ways.

In one aspect, the compositions can be prepared by adding cyclodextrins to optionally buffered aqueous solution; adding benzimidazole derivatives or pharmaceutically acceptable salts thereof; optionally adding other excipients; and then adding sufficient water to make up the final volume. If necessary, the pH can be readjusted to achieve the desired pH range.

By way of another example, the compositions can be prepared by adding cyclodextrins and benzimidazole derivatives or pharmaceutically acceptable salts thereof to optionally buffered water solution; adjusting the pH to achieve the desired pH range; optionally adding other excipients; and then adding sufficient water to make up the final volume.

By way of another example, the compositions can be prepared by adding benzimidazole derivatives or pharmaceutically acceptable salts thereof to optionally buffered water solution; adding cyclodextrins; adding a pH adjuster to achieve the desired pH; optionally adding other excipients; and then adding sufficient water to make up the final volume.

Methods for making a ready-to-use aqueous pantoprazole composition suitable for parenteral administration may comprise the steps of providing an effective amount of pantoprazole in a solution comprising at least one cyclodextrin and optionally a buffer. If required, the pH of the solution can be adjusted using a suitable pH adjuster. The compositions are dispensed in pharmaceutically acceptable containers for storage. In one aspect, the compositions are dispensed in pharmaceutically acceptable containers for storage and direct administration to patients.

Methods for making a precipitate free benzimidazole formulation suitable for parenteral administration comprise steps of providing an effective amount of benzimidazole derivative and at least one cyclodextrin and optionally a buffer in a solution. If required, the pH of the solution can be adjusted using a suitable pH adjuster. The compositions are dispensed in pharmaceutically acceptable containers for storage. In one aspect, compositions are dispensed in pharmaceutically acceptable containers for storage and direct administration to patients.

In one aspect, the composition of pantoprazole is made by adding pantoprazole and cyclodextrins in aqueous solution optionally containing a buffer, adjusting the pH of the solution to achieve desired pH, and optionally adding other excipients.

In one aspect, the aqueous pharmaceutical solution of pantoprazole is made by following steps: adding cyclodextrins into aqueous solution optionally containing a buffer, adjusting pH of the solution to achieve desired pH, adding pantoprazole, and optionally adding other excipients.

In one aspect, the aqueous pharmaceutical solution of pantoprazole is made by following steps: adding other excipients into aqueous solution optionally containing a buffer, adjusting pH of the solution to achieve desired pH, adding cyclodextrins and pantoprazole.

The pharmaceutical compositions can be packaged for use in a variety of containers. The compositions may be packaged in a pharmaceutically acceptable container, such as vial, ampoule, capsule, sachet, an intravenous bag, or bottle.

In one aspect, concentration of dissolved oxygen in the solution is not more than 2 ppm. In one aspect, compositions are packaged under nitrogen atmosphere.

Examples of suitable procedures for producing sterile pharmaceutical drug products include, but are not limited to, terminal moist heat sterilization, ethylene oxide, radiation (i.e., gamma and electron beam), and aseptic processing techniques.

### EXAMPLES

Examples in the present disclosure are intended to be illustrative and not limiting as to the general disclosure.

### General Techniques

All formulations presented in examples below were prepared with the starting concentrations of pantoprazole and cyclodextrins. Some of formulations comprised other excipients or buffers which are stated in corresponding tables. Prepared compositions were transferred into containers, such as vials, to achieve desirable amount of active component per container.

Unless otherwise stated, in examples prepared with HPBCD, molar substitution of HPBCD's was in range from 0.6 to 1.0.

Formulations of active component without cyclodextrins are shown for comparative purposes.

### Analytical Procedures

After preparation, initial time point level of active pharmaceutical ingredient and impurities were determined by UPLC and afterwards containers were loaded to stability chambers at different storage conditions, 40° C., 30° C. and 25° C.

In order to determine stability of active pharmaceutical ingredient in formulations according to the present disclosure, containers were taken from stability chambers at various time points, such as 3 days, 7 days, 14 days, 21 days, 1 month, 2 months, 3 months, 4 months, 6 months etc. and analyzed by UPLC.

Assay and impurities for active pharmaceutical ingredient were determined by UPLC analysis, equipped with an ultraviolet (UV) detector. All samples were analyzed using a reverse-phase C18 column and by measuring the absorbance (area under the curve) at a wavelength of 290 and 305 nm. The UPLC conditions used were those disclosed below.

### Assay Procedure

### Chromatographic System:

Mode: LC
Detector: UV 290 nm
Column: 2.1-mm; 15-cm; 2.5 μm packing L1, Stationary phase: octadecylsilyl silica gel for chromatography R
Column temperature: 30° C.
Autosampler: 5° C.
Flow rate: 0.5 mL/min
Injection volume: 3 μL
Diluent: water
Buffer: 10 mM potassium phosphate adjusted with phosphoric acid to a pH of 7.00±0.05
Solution A: Buffer and water (90:10)
Solution B: Buffer and water (50:50)

### Mobile Phase—See Table:

| Time (min) | Solution A (%) | Solution B (%) |
|---|---|---|
| 1 | 96 | 4 |
| 10 | 10 | 90 |
| 12 | 96 | 4 |
| 15 | 96 | 4 |

Sample solution preparation for formulation: 0.1 mg/mL (e.g., 2 mL of the formulation is dissolved and diluted to 20.0 mL with diluent, then additionally in second step 2.5 mL of previously prepared sample (in the first dilution step) diluted to 10.0 mL with diluent)

Sample solution preparation for standard solution: 0.1 mg/mL of USP Pantoprazole Sodium RS

### Impurities Procedure

### Chromatographic System:

Mode: LC
Detector: UV 290 and 305 nm
Column: 2.1-mm; 15-cm; 2.5 μm packing L1, Stationary phase: octadecylsilyl silica gel for chromatography R
Column temperature: 30° C.
Autosampler: 5° C.
Flow rate: 0.5 mL/min
Injection volume: 3 μL
Diluent: water
Buffer: 10 mM potassium phosphate adjusted with phosphoric acid to a pH of 7.00±0.05
Solution A: Buffer and water (90:10)
Solution B: Buffer and water (50:50)

### Mobile Phase—See Table:

| Time (min) | Solution A (%) | Solution B (%) |
|---|---|---|
| 1 | 96 | 4 |
| 23 | 30 | 70 |
| 30 | 2 | 98 |
| 33 | 2 | 98 |
| 35 | 96 | 4 |
| 40 | 96 | 4 |

Sample solution: 0.4 mg/mL (e.g. 2 mL of the formulation is dissolved and diluted to 20.0 mL with diluent)

### Calculation of the Assay of Active Compound

The assay of the active compound is calculated using the following equation:

$$Assay ( \% ) = \frac{Amps}{Ampstd} \times \frac{c s t d}{c s} \times 100 \%$$

where:
Amps=peak area of the Pantoprazole peak in Sample solution
Ampstd=peak area of the Pantoprazole peak in Standard solution
cs=concentration of Pantoprazole Sodium in the Sample solution (mg/mL)
cstd=concentration of Pantoprazole Sodium in the Standard solution (mg/mL)

### Calculation of the Assay Drop of the Main Peak

Assay(tp) (%)—Value of the assay of the main peak at time point (tp) different than initial, for example: 7 days, 1 month, 2 months etc. at different storage conditions such as 25° C., 40° C., determined by UPLC

Assay(st) (%)—Value of the assay of the main peak at initial time point

ΔAssay—Calculated assay drop of the main compound: (%)

$$Δ Assay ( \% ) = Assay ( st ) ( \% ) - Assay ( tp ) ( \% )$$

### Calculation of the Individual Impurity

The content of impurities monitored at 290 nm, is given as area % of the total area, calculated using the following equation:

$$Impurity ( \% ) = \frac{A i}{A t o t} \times 100 \%$$

where:
area %=area % of an individual peak
Ai=peak area of an individual peak
Atot=total sample peak area

### Visible Particle Inspection

At the beginning of visual inspection at each stability time point, the bottom of the container is checked for precipitate formation, then visual particle inspection is checked by gently swirling and inverting containers, ensuring that no air bubbles are produced. Containers were inspected for approximately 15 sec with naked eye and under the magnifier (having 1.7× magnification, Adelphi Apollo I Liquid Viewer). Observation regarding visual inspection were noted for inspected samples.

### LIST OF ABBREVIATIONS IN TABLES

API—Active pharmaceutical ingredient
HPBCD—hydroxypropyl beta cyclodextrin
PNT—pantoprazole sodium sesquihydrate
SBECD—sulfobutyl ether cyclodextrin
ARG—Arginine
Y/N—Yes/No

### Example 1

In order to show the impact of cyclodextrins on pantoprazole chemical and physical stability in composition, below experiments are conducted and showed herein.

Aqueous formulation of pantoprazole sodium sesquihydrate and cyclodextrin were prepared with the starting concentrations of pantoprazole and cyclodextrins in aqueous solution, adjusting the pH of the solution to achieve the desired pH. Some of the formulations comprised other excipients or buffers, which concentration or molar ratios are stated in corresponding tables. Prepared compositions were transferred into containers, such as vials, to achieve desirable amount of active component per container.

TABLE 1
PHYSICAL STABILITY OF AQUEOUS
FORMULATIONS OF PANTOPRAZOLE AT A PH OF 10.5

| | Other | | Stability | Precipitation |
|---|---|---|---|---|
| (API) | ingredients | Temp. | time point | (Y/N) |
| PNT 4 | Carbonate/bicarbonate | 40° C. | Start | N |
| mg/ml | buffer (20 mM) | | 7 days | Y |
| | | 25° C. | 1 M | Y |
| PNT 4 | Phosphate buffer | 40° C. | Start | N |
| mg/ml | (100 mM) | | 7 days | Y |
| | | 25° C. | 1 M | Y |
| PNT 4 | Carbonate/bicarbonate | 40° C. | Start | N |
| mg/ml | buffer (20 mM) | | | |
| | HPβCD in molar ratio | | 7 days | N |
| | to API 3:1 | 25° C. | 6 M | N |
| PNT 4 | Carbonate/bicarbonate | 40° C. | Start | N |
| mg/ml | buffer (20 mM) | | | |
| | SBEβCD in molar ratio | | 7 days | N |
| | to API 3:1 | 25° C. | 5 M | N |
| PNT 4 | Carbonate/bicarbonate | 40° C. | Start | N |
| mg/ml | buffer (100 mM) | | | |
| | SBEβCD in molar ratio | | 7 days | N |
| | to API 1:1 | 25° C. | 6 M | N |
| PNT 4 | Carbonate/bicarbonate | 40° C. | Start | N |
| mg/ml | buffer (20 mM) | | | |
| | HPβCD in molar ratio | | 7 days | N |
| | to API 2:1 | 25° C. | 2 M | N |
| PNT 4 | Phosphate buffer | 40° C. | Start | N |
| mg/ml | (100 mM) | | | |
| | HPβCD in molar ratio | | 7 days | N |
| | to API 2:1 | 25° C. | 2 M | N |

### Example 2

TABLE 2
CHEMICAL AND PHYSICAL STABILITY OF AQUEOUS FORMULATIONS
COMPRISING PANTOPRAZOLE AND DIFFERENT CYCLODEXTRINS AT A PH OF 10.5

| Active | | | | | | | |
|---|---|---|---|---|---|---|---|
| pharmaceutical | | | Stability | | Pantoprazole | | |
| ingredient | Other | | time | Pantoprazole | Assay Drop | Impurity | Precipitation |
| (API) | ingredients | Temp. | point | Assay (%) | (%) | A (%) | (Y/N) |
| PNT 4 mg/ml | Carbonate/mM) | 40° C. | Start | 99.3 | 1 | <0.05 | N |
| | bicarbonate | | 7 days | 95.7 | 3.6 | 0.07* | Y |
| | buffer (20 mM) | 25° C. | 3M | 89.0 | 10.3 | 0.42* | Y |
| PNT 4 mg/ml | Carbonate/ | 40° C. | Start | 99.6 | / | <0.05 | N |
| | bicarbonate | | 7 days | 96.6 | 3.0 | <0.05 | N |
| | buffer | 25° C. | 3M | 91.3 | 8.3 | 0.07 | N |
| | (20 mM) | | | | | | |
| | HPβCD in | | | | | | |
| | molar ratio | | | | | | |
| | to API 3:1 | | | | | | |
| PNT 4 mg/ml | Phosphate | 40° C. | Start | 97.8 | / | <0.05 | N |
| | buffer | | 7 days | 94.6 | 3.2 | 0.16* | Y |
| | (100 mM) | | | | | | |
| PNT 4 mg/ml | Phosphate | 40° C. | Start | 97.7 | / | <0.05 | N |
| | buffer | | 7 days | 95.3 | 2.4 | <0.05 | N |
| | (100 mM) | | | | | | |
| | HPβCD in | | | | | | |
| | molar ratio | | | | | | |
| | to API 2:1 | | | | | | |

*Due to precipitate formation, Impurity A value is expressed only for supernatant.

### Example 3

Table 3 shows chemical stability of aqueous formulations comprising pantoprazole, cyclodextrins and additional excipients at a pH of 10.5. All formulations in Table 3 were clear at measured time-points, without any visible particles.

TABLE 3
CHEMICAL STABILITY OF AQUEOUS FORMULATIONS COMPRISING
PANTOPRAZOLE, CYCLODEXTRINS AND ADDITIONAL EXCIPIENTS AT A PH OF 10.5

| Active | | | | | | |
|---|---|---|---|---|---|---|
| pharmaceutical | | | Stability | | Pantoprazole | |
| ingredient | | | time | Pantoprazole | Assay Drop | Impurtiy A |
| (API) | Other ingredients | Temp. | point | Assay (%) | (%) | (%) |
| PNT 4 mg/ml | Carbonate/bicarbonate | 40° C. | Start | 99.7 | / | <0.05 |
| | buffer (20 mM) | | 7 days | 96.1 | 3.6 | <0.05 |
| | Arg in molar ratio to | | | | | |
| | API 5:1 | | | | | |
| | HPβCD in molar ratio | 25° C. | 3M | 92.0 | 7.7 | <0.05 |
| | to API 3:1 | | | | | |
| PNT 4 mg/ml | Carbonate/bicarbonate | 40° C. | Start | 98.0 | 1 | <0.05 |
| | buffer (20 mM) | | 7 days | 95.3 | 2.7 | <0.05 |
| | Arg in molar ratio to | | | | | |
| | API 5:1 | | | | | |
| | HPβCD in molar ratio | 25° C. | 3M | 93.6 | 4.4 | <0.05 |
| | to API 6:1 | | | | | |
| PNT 4 mg/ml | Carbonate/bicarbonate | 40° C. | Start | 100.0 | / | <0.05 |
| | buffer (20 mM) | | 7 days | 95.9 | 4.1 | <0.05 |
| | Arg in molar ratio to | | | | | |
| | API 5:1 | | | | | |
| | HPβCD in molar ratio | 25° C. | 3M | 92.8 | 7.2 | <0.05 |
| | to API 2:1 | | | | | |
| PNT 4 mg/ml | Carbonate/bicarbonate | 40° C. | Start | 99.0 | / | <0.05 |
| | buffer (20 mM) | | 7 days | 95.5 | 3.5 | <0.05 |
| | Arg in molar ratio to | | | | | |
| | API 10:1 | | | | | |
| | HPβCD in molar ratio | 25° C. | 3M | 93.9 | 5.1 | <0.05 |
| | to API 2:1 | | | | | |
| PNT 4 mg/ml | Carbonate/bicarbonate | 40° C. | Start | 99.0 | 1 | <0.05 |
| | buffer (50 mM) | | 7 days | 96.4 | 2.6 | <0.05 |
| | Arg in molar ratio to | | | | | |
| | API 5:1 | | | | | |
| | HPβCD in molar ratio | 25° C. | 3M | 92.9 | 6.1 | <0.05 |
| | to API 3:1 | | | | | |
| PNT 4 mg/ml | Carbonate/bicarbonate | 40° C. | Start | 99.7 | / | <0.05 |
| | buffer (80 mM) | | 7 days | 96.4 | 3.3 | <0.05 |
| | Arg in molar ratio to | | | | | |
| | API 5:1 | | | | | |
| | HPβCD in molar ratio | 25° C. | 3M | 93.1 | 6.6 | <0.05 |
| | to API 2:1 | | | | | |
| PNT 4 mg/ml | Carbonate/bicarbonate | 40° C. | Start | 99.0 | 1 | <0.05 |
| | buffer (100 mM) | | 7 days | 96.1 | 2.9 | <0.05 |
| | Arg in molar ratio to | | | | | |
| | API 5:1 | | | | | |
| | HPβCD in molar ratio | 25° C. | 3M | 93.7 | 5.3 | <0.05 |
| | to API 3:1 | | | | | |
| PNT 4 mg/ml | Carbonate/bicarbonate | 40° C. | Start | 99.6 | 1 | <0.05 |
| | buffer (50 mM) | | 7 days | 96.6 | 3.0 | <0.05 |
| | Arg in molar ratio to | | | | | |
| | API 1:1 | | | | | |
| | HPβCD in molar ratio | 25° C. | 3M | 93.1 | 6.5 | <0.05 |
| | to API 6:1 | | | | | |
| PNT 4 mg/ml | Carbonate/bicarbonate | 40° C. | Start | 98.6 | / | <0.05 |
| | buffer (50 mM) | | 7 days | 95.5 | 3.1 | <0.05 |
| | Arg in molar ratio to | | | | | |
| | API 5:1 | | | | | |
| | HPβCD in molar ratio | 25° C. | 4M | 90.1. | 8.5 | <0.05 |
| | to API 8.5:1 | | | | | |

### Example 4

Table 4 shows chemical stability of aqueous formulations comprising pantoprazole, cyclodextrins and additional excipients at various pHs. All formulations in Table 4 were clear at measured time-points, without any visible particles.

TABLE 4
CHEMICAL STABILITY OF AQUEOUS FORMULATIONS COMPRISING
PANTOPRAZOLE, CYCLODEXTRINS AND ADDITIONAL EXCIPIENTS AT VARIOUS PHS

| Active | | | | | | | |
|---|---|---|---|---|---|---|---|
| pharmaceutical | | | | Stability | | Pantoprazole | |
| ingredient | | | | time | Pantoprazole | Assay Drop | Impurity |
| (API) | Other ingredients | pH | Temp. | point | Assay (%) | (%) | A (%) |
| PNT 4 mg/ml | Carbonate/bicarbonate | 11.5 | 40° C. | Start | 99.2 | / | <0.05 |
| | buffer (50 mM) | | | 7 days | 98.0 | 1.2 | <0.05 |
| | Arg in molar | | | | | | |
| | ratio to API 5:1 | | | | | | |
| | HPβCD in molar | | 25° C. | 4M | 95.9 | 3.3. | <0.05 |
| | ratio to API 6:1 | | | | | | |
| PNT 4 mg/ml | Carbonate/bicarbonate | 11.5 | 40° C. | Start | 99.1 | / | <0.05 |
| | buffer (50 mM) | | | 7 days | 97.9 | 1.2 | <0.05 |
| | Arg in molar | | | | | | |
| | ratio to API 5:1 | | | | | | |
| | HPβCD in molar | | 25° C. | 4M | 95.4 | 3.7 | <0.05 |
| | ratio to API 3:1 | | | | | | |
| PNT 4 mg/ml | Carbonate/bicarbonate | 11.2 | 40° C. | Start | 99.4 | / | <0.05 |
| | buffer (50 mM) | | | 7 days | 96.5 | 2.9 | <0.05 |
| | Arg in molar | | | | | | |
| | ratio to API 5:1 | | | | | | |
| | HPβCD in molar | | | | | | |
| | ratio to API 6:1 | | | | | | |
| PNT 4 mg/ml | Carbonate/bicarbonate | 12.0 | 40° C. | Start | 98.4 | / | <0.05 |
| | buffer (50 mM) | | | 7 days | 96.5 | 1.9 | <0.05 |
| | Arg in molar | | | | | | |
| | ratio to API 5:1 | | | | | | |
| | HPβCD in molar | | | | | | |
| | ratio to API 3:1 | | | | | | |
| PNT 4 mg/ml | Phosphate buffer | 11.0 | 40° C. | Start | 98.8 | 1 | <0.05 |
| | (148 mM) | | | 7 days | 96.3 | 2.5 | <0.05 |
| | Arg in molar | | | | | | |
| | ratio to API 5:1 | | | | | | |
| | HPβCD in molar | | 25° C. | 6M | 90.8 | 8.0 | <0.05 |
| | ratio to API 3:1 | | | | | | |
| PNT 4 mg/ml | Phosphate buffer | 11.0 | 40° C. | Start | 98.2 | / | <0.05 |
| | (100 mM) | | | 7 days | 95.7 | 2.5 | <0.05 |
| | Arg in molar | | | | | | |
| | ratio to API 5:1 | | | | | | |
| | HPβCD in molar | | | | | | |
| | ratio to API 3:1 | | | | | | |
| PNT 4 mg/ml | Phosphate buffer | 11.5 | 40° C. | Start | 99.7 | / | <0.05 |
| | (100 mM) | | | 7 days | 97.0 | 2.7 | <0.05 |
| | Arg in molar | | | | | | |
| | ratio to API 5:1 | | | | | | |
| | HPβCD in molar | | 25° C. | 4M | 94.6 | 5.2 | <0.05 |
| | ratio to API 3:1 | | | | | | |
| PNT 4 mg/ml | Carbonate/bicarbonate | 11.0 | 40° C. | Start | 98.7 | 1 | <0.05 |
| | buffer (50 mM) | | | 7 days | 96.2 | 2.5 | <0.05 |
| | Arg in molar | | | | | | |
| | ratio to API 5:1 | | | | | | |
| | HPβCD in molar | | 25° C. | 4M | 93.1 | 5.6 | <0.05 |
| | ratio to API 6:1 | | | | | | |
| PNT 4 mg/ml | Carbonate/bicarbonate | 11.0 | 40° C. | Start | 99.3 | / | <0.05 |
| | buffer (50 mM) | | | 7 days | 97.2 | 2.1 | <0.05 |
| | Arg in molar | | | | | | |
| | ratio to API 5:1 | | | | | | |
| | HPβCD in molar | | 25° C. | 4M | 94.6 | 4.7 | <0.05 |
| | ratio to API 3:1 | | | | | | |

### Example 5

Liquid formulations of various benzimidazole derivatives in accordance with the present disclosure are prepared with the starting concentrations of benzimidazole derivative and cyclodextrins in aqueous solution. pH of the solution is adjusted to achieve desired pH. Some of formulations comprises other excipients or buffers. Compositions are transferred into containers, such as vials, to achieve desirable amount of active component per container.

It is expected that the formulations are stable at least for 7 days at 40° C. and for at least 3 months at 25° C., and that this can be extrapolated to indicate that the formulations are stable or stabilized for up to about 24 months.

### Example 6

The following numbered items represent embodiments of liquid pharmaceutical formulations comprising active component.

Item 1. A liquid pantoprazole composition having a pH in the range of 8.5-12, wherein the composition comprises:
pantoprazole or its pharmaceutically acceptable salt;
optionally at least one buffer capable of maintaining the pH of the composition from 8.5 to 12; and
at least one cyclodextrin.

Item 2. The composition of claim 1, where pantoprazole is in form of pantoprazole sodium sesquihydrate.

Item 3. The composition of item 1, wherein the composition is free of particles.

Item 4. The composition of item 1, wherein the composition is free of particles for at least 7 days at 40° C.

Item 5. The composition of item 1, where the pantoprazole and at least one cyclodextrin are in a molar ratio of 1:1 to 1:14.

Item 6. The composition of item 1, where the cyclodextrin is a beta cyclodextrin selected from HPBCD or SBECD.

Item 7. The composition of item 1, wherein the composition is an aqueous composition.

Item 8. The composition of item 1, where composition is a ready-to-use composition.

Item 9. The composition of item 1, where a concentration of pantoprazole or its pharmaceutically acceptable salt is from 0.2 mg/ml to 10 mg/ml.

Item 10. The composition of item 1, where the composition comprise at least one amino acid or its pharmaceutically acceptable salt.

Item 11. The composition of item 10, where amino acid is arginine, tryptophane, phenylalanine, tyrosine, proline, taurine, lysine, histidine, glutamine, glutamate, or a pharmaceutically acceptable salt or derivative thereof.

Item 12. The composition of item 10, where the amino acid is arginine or its pharmaceutically acceptable salt or derivative.

Item 13. The composition of item 10, where the pantoprazole and at least one amino acid are in a molar ratio of 1:0.5 to 1:20.

Item 14. The composition of item 1, where the pH of the composition is from 10.5 to 12.0.

Item 15. The composition of item 1, wherein the buffer is selected from phosphate, carbonate, meglumine, ammonium, glycine, and borate.

Item 16. The composition of item 15, wherein the at least one buffer is carbonate/bicarbonate buffer.

Item 17. The composition of item 15, wherein the at least one buffer is phosphate buffer.

Item 18. The composition of item 15, wherein the at least one buffer has a concentration from 20 mM to 200 mM.

Item 19. The composition of item 1, wherein the composition is comprised in a unit dosage form.

Item 20. A method of treatment of gastroesophageal reflux disease (GERD) associated with a history of Erosive Esophagitis and/or pathological hypersecretion conditions including Zollinger-Ellison (ZE) Syndrome comprising administration of the composition of pantoprazole according to item 1 to the mammal in need thereof.

Item 21. A method of producing the pantoprazole composition according to item 1, comprising the steps of providing an effective amount of pantoprazole or its pharmaceutically acceptable salt in a solution comprising at least one cyclodextrin and optionally a buffer.

Item 22. An aqueous composition having a pH in the range 8.5-12 and where the aqueous composition comprise:
pantoprazole or a pharmaceutically acceptable salt thereof;
optionally one or more buffers having at least one ionizable group with a pKa in the range 7.0 to 14 and which pKa is within 2 pH units of the pH of the composition; and
at least one beta cyclodextrin;
wherein the pantoprazole is present in the composition at a total concentration of 0.4 mg/ml to 4 mg/ml.

Item 23. The aqueous composition of item 22, where the beta cyclodextrin is HPBCD and wherein the pantoprazole and HPBCD are in a molar ratio of 1:3 to 1:6.

Item 24. The aqueous composition of item 22, where the composition further comprises arginine or its pharmaceutically acceptable salt.

Item 25. The aqueous composition of item 24, wherein the beta cyclodextrin is HPBCD, wherein pantoprazole and HPBCD are in a molar ratio of 1:3 to 1:6, wherein pantoprazole and arginine are in molar ratio of 1:2 to 1:6, and wherein the buffer is a bicarbonate buffer.

Item 26. The aqueous composition of item 25, wherein the pH of the composition is from 10.5 to 12.

Item 27. A method of treatment of gastroesophageal reflux disease (GERD) associated with a history of Erosive Esophagitis and/or pathological hypersecretion conditions including Zollinger-Ellison (ZE) Syndrome comprising administration of composition of pantoprazole according to item 1 or 22 to the mammal in need thereof, wherein the administration does not involve reconstitution of a lyophilized composition of pantoprazole.

1. A benzimidazole derivative composition having a pH in the range of 8.5-12, wherein the composition comprises:
a benzimidazole derivative;
at least one cyclodextrin; and
optionally at least one buffer capable of maintaining the pH of the composition from 8.5 to 12.

2. The composition of claim 1, wherein composition is free of particles.

3. The composition of claim 1, wherein composition is free of particles for at least 7 days at 40° C.

4. The composition of claim 1, where benzimidazole derivative and at least one cyclodextrin are in a molar ratio of 1:1 to 1:14.

5. The composition of claim 1, where cyclodextrin is a beta cyclodextrin selected from HPBCD or SBECD.

6. The composition of claim 1, wherein the composition is a liquid.

7. The composition of claim 1, where composition is a ready-to-use composition.

8. The composition of claim 1, where benzimidazole derivative comprises omeprazole, lansoprazole, esomeprazole, dexlansoprazole, rabeprazole, pantoprazole, or pharmaceutically acceptable salts thereof.

9. The composition of claim 8, where benzimidazole derivative is pantoprazole.

10. The composition of claim 1, where concentration of the benzimidazole derivative or its pharmaceutically acceptable salt is from 0.2 mg/ml to 10 mg/ml.

11. The composition of claim 1, where composition comprises at least one amino acid or its pharmaceutically acceptable salt.

12. The composition of claim 11, where amino acid comprises arginine, tryptophane, phenylalanine, tyrosine, proline, taurine, lysine, histidine, glutamine, glutamate, or a pharmaceutically acceptable salt or derivative thereof.

13. The composition of claim 11, where benzimidazole derivative or its pharmaceutically acceptable salt and at least one amino acid are in a molar ratio of 1:0.5 to 1:20.

14. The composition of claim 1, where the pH of the composition is from 10.5 to 12.0.

15. The composition of claim 1, wherein the buffer is selected from phosphate, carbonate, meglumine, ammonium, glycine, and borate.

16. The composition of claim 12, wherein at least one buffer is a carbonate/bicarbonate buffer.

17. The composition of claim 12, wherein at least one buffer is a phosphate buffer.

18. The composition of claim 12, wherein at least one buffer has a concentration from 20 mM to 200 mM.

19. The aqueous composition of claim 1, where composition is comprised in a unit dosage form.

20. A method of treating gastroesophageal reflux disease (GERD) associated with a history of Erosive Esophagitis and/or pathological hypersecretion conditions including Zollinger-Ellison (ZE) Syndrome comprising administering the composition according to claim 8 to a mammal in need thereof.

21. A method of producing a benzimidazole derivative composition according to claim 1, comprising the steps of providing an effective amount of the benzimidazole derivative or its pharmaceutically acceptable salt in a solution comprising at least one cyclodextrins and optionally a buffer.
